(19) United States
(12) Patent Application Publication
(10) Pub. No.: US 2024/0398887 A1
(43) Pub. Date: Dec. 5, 2024

(54) TRADITIONAL CHINESE MEDICINE COMPOSITE FOR REPLENISHING QI AND NOURISHING BLOOD, AND PREPARATION METHOD AND USE THEREOF

(71) Applicant: Beijing Chuanglian Huitong Biotechnology Co., Ltd., Beijing (CN)

(72) Inventor: Jing Wang, Beijing (CN)

(21) Appl. No.: 18/799,967

(22) Filed: Aug. 9, 2024

(30) Foreign Application Priority Data
Aug. 11, 2023 (CN) 2023110123399

(51) Int. Cl. A61K 36/88; A61K 36/232; A61K 36/258; A61K 36/80

(52) U.S. Cl. CPC A61K 36/88; A61K 36/232; A61K 36/258; A61K 36/80

(57) ABSTRACT

The present invention discloses a traditional Chinese medicine composition for tonifying qi and nourishing blood. The traditional Chinese medicine composition includes, by mass, the following components: 8-12 parts of saffron, 200-300 parts of Chinese angelica, 200-300 parts of ginseng, 200-300 parts of pipewort, and 200-300 parts of butterflybush flowers. The traditional Chinese medicine composition is used for eye care and health protection. The traditional Chinese medicine composite is applicable to symptoms including: dry eyes, astringent eyes, itchy eyes, eyestrain, foreign body sensation, pterygium, blurred vision, photophobia, epiphora induced by wind, macular degeneration, prevention of myopia, conjunctival concretion, and cataracts. The traditional Chinese medicine composition for tonifying qi and nourishing blood provided by the present invention is extracted from pure traditional Chinese medicines without hormones or irritation and is used by means of atomizing with cool feeling; the provided traditional Chinese medicine composition is capable of dredging meridians around an eye, improving a blood supply of the eye, removing congestion, tonifying qi, and nourishing blood, thus improving an self-healing ability of eye tissue and repairing a damaged optic nerve. On the basis of practice of 5,000-year-long traditional Chinese medicine ophthalmology culture, the inventor believes that the eye can see things clearly based on enriched blood. The provided traditional Chinese medicine composition has advantages of being simple, comfortable and convenient to use.

### TECHNICAL FIELD

The present invention relates to the field of traditional Chinese medicines, in particular a traditional Chinese medicine composite for replenishing qi and nourishing blood, and a preparation method and use thereof.

### BACKGROUND

An eye is an integral part of a body, and many systemic diseases may be manifested in the eye. In order to “capture” a message of light, the eye must be exposed to a surface of the body, which increases the chances of trauma and external pathogenic invasion. Eye diseases will eventually affect a visual function. An impairment of the visual function not only makes a patient suffer, but also brings misfortune to a family and society, so eye care and health protection are very important. Most of existing eye care and health protection methods use Western medicines, which has great side effects. Or a physiotherapy mode is used, which is slow to work. In order to overcome defects of the prior art, it is necessary to design a traditional Chinese medicine composite for replenishing qi and nourishing blood.

### SUMMARY

In order to overcome defects of the prior art, a traditional Chinese medicine composite for replenishing qi and nourishing blood, and a preparation method and use thereof are provided.

The present disclosure is implemented through the following solutions.

A traditional Chinese medicine composite for replenishing qi and nourishing blood includes, by mass, the following components: 8-12 parts of saffron, 200-300 parts of angelica, 200-300 parts of ginseng, 200-300 parts of pipewort, and 200-300 parts of butterflybush flowers.

The traditional Chinese medicine composite includes, by mass, the following components: 12 parts of saffron, 250 parts of angelica, 200 parts of ginseng, 300 parts of pipewort, and 250 parts of butterflybush flowers.

The traditional Chinese medicine composite includes, by mass, the following components: 8 parts of saffron, 300 parts of angelica, 250 parts of ginseng, 200 parts of pipewort, and 300 parts of butterflybush flowers.

The traditional Chinese medicine composite includes, by mass, the following components: 10 parts of saffron, 200 parts of angelica, 300 parts of ginseng, 250 parts of pipewort, and 200 parts of butterflybush flowers.

A preparation method of a traditional Chinese medicine composite, wherein the method includes the following steps: weighing, by mass, 8-12 parts of saffron, 200-300 parts of angelica, 200-300 parts of ginseng, 200-300 parts of pipewort, and 200-300 parts of butterflybush flowers, putting the components into a distiller, adding water to soak the components for 2 hours, and distilling a medical solution; adding water, and then performing re-distilling to distill a medical solution; then again adding water and sodium chloride to distill a medical solution; and mixing the medical solutions of 3 times to obtain a finished traditional Chinese medicine composite.

According to an application of a traditional Chinese medicine composite, the traditional Chinese medicine composite is used for eye care and health protection.

The traditional Chinese medicine composite is applicable to symptoms including: dry eyes, astringent eyes, itchy eyes, eyestrain, foreign body sensation, pterygium, blurred vision, photophobia, epiphora induced by wind, macular degeneration, prevention of myopia, eye stones, and cataracts.

The present disclosure has the beneficial effects:

The traditional Chinese medicine composite for replenishing qi and nourishing blood provided by the present disclosure is extracted from pure traditional Chinese medicines without hormones or irritation. The use of an atomized cold spray may dredge meridians around an eye, improve a blood supply of the eye, remove congestion, replenish qi, and nourish blood, thus improving an self-healing ability of eye tissue, and repairing a damaged optic nerve. 5,000 years of traditional Chinese medicine ophthalmology culture is practiced, “the eye can see when it is nourished by blood”, and the use is simple, comfortable, and fast.

### BRIEF DESCRIPTION OF DRAWINGS

Preferred embodiments of the present disclosure are further described below.

A traditional Chinese medicine composite for replenishing qi and nourishing blood includes, by mass, the following components: 8-12 parts of saffron, 200-300 parts of angelica, 200-300 parts of ginseng, 200-300 parts of pipewort, and 200-300 parts of butterflybush flowers.

The traditional Chinese medicine composite includes, by mass, the following components: 12 parts of saffron, 250 parts of angelica, 200 parts of ginseng, 300 parts of pipewort, and 250 parts of butterflybush flowers.

The traditional Chinese medicine composite includes, by mass, the following components: 8 parts of saffron, 300 parts of angelica, 250 parts of ginseng, 200 parts of pipewort, and 300 parts of butterflybush flowers.

The traditional Chinese medicine composite includes, by mass, the following components: 10 parts of saffron, 200 parts of angelica, 300 parts of ginseng, 250 parts of pipewort, and 200 parts of butterflybush flowers.

A preparation method of a traditional Chinese medicine composite, wherein the method includes the following steps: 8-12 parts of saffron, 200-300 parts of angelica, 200-300 parts of ginseng, 200-300 parts of pipewort, and 200-300 parts of butterflybush flowers are weighed by mass; the components are put into a distiller, water is added to soak the components for 2 hours, and a medical solution is distilled; water is added, and then re-distilling is performed to distill a medical solution; then again water and sodium chloride are added to distill a medical solution; and the medical solutions of 3 times are mixed to obtain a finished traditional Chinese medicine composite.

According to an application of a traditional Chinese medicine composite, the traditional Chinese medicine composite is used for eye care and health protection. The traditional Chinese medicine composite is applicable to symptoms including: dry eyes, astringent eyes, itchy eyes, eyestrain, foreign body sensation, pterygium, blurred vision, photophobia, epiphora induced by wind, macular degeneration, prevention of myopia, eye stones, and cataracts.

The present application is further illustrated below in conjunction with examples.

Eye stones: the traditional Chinese medicine composite in the present application was put into a spray instrument 6-8 times a day and for 30 seconds each time. Spraying was performed in the morning and evening and randomly during the day. 10 days was a course of treatment. Patients were cured within 2-5 courses. A total of 12 people were treated, and all were cured.

Macula lutea: the traditional Chinese medicine composite in the present application is put into a spray instrument 6-8 times a day and for 30 seconds each time. Spraying must be performed in the morning and evening and randomly during the day. A mild symptom was eliminated in 3 weeks. The traditional Chinese medicine composite was used until there was no symptom at all. A total of 38 people were treated, and all were cured.

Photophobia: the traditional Chinese medicine composite in the present application was put into a spray instrument 6-8 times a day and for 30 seconds each time. Spraying must be performed in the morning and evening and randomly during the day. 10 days was a course of treatment. Patients were cured within 2-5 courses. A total of 26 people were treated, and all were cured.

Li, female, 56 years old, in March 2018 in the Chinese Medicine Hall of the Seventh Hospital of Handan City, had dry eyes and a foreign body sensation. The patient was treated for 3 courses. After the second course, the patient recovered basically without discomfort, and was cured for another course. Du Xiulan, female, 48 years old, in March 2018 in the Chinese Medicine Hall of the Seventh Hospital of Handan City. The patient had more than one year of discomfort in eye blinking, and had white granules on upper and lower eyelids. Through 6 courses of medication, the small particles disappeared, and she recovered.

The traditional Chinese medicine composite in the present application is extracted and purified from Chinese herbal medicines with zero addition and without hormones or irritation, only needs to be sprayed, and is easy to use and carry around.

Although the technical solutions of the present disclosure have been described and illustrated in details, it should be understood that modifications and equivalents of the above-described embodiments will be apparent to those skilled in the art, and these modifications or improvements made on the basis of not deviating from the spirit of the present disclosure belong to the scope of protection claimed by the present disclosure.

What is claimed is:

1. A traditional Chinese medicine composition for tonifying qi and nourishing blood, comprising, by mass, the following components: 8-12 parts of saffron, 200-300 parts of Chinese angelica, 200-300 parts of ginseng, 200-300 parts of pipewort, and 200-300 parts of butterflybush flowers; wherein
a preparation method of the traditional Chinese medicine composition comprises the following steps: weighing, by mass, 8-12 parts of saffron, 200-300 parts of Chinese angelica, 200-300 parts of ginseng, 200-300 parts of pipewort, and 200-300 parts of butterflybush flowers, putting the components into a still, adding water to soak the components for 2 hours, and distilling a medical solution; adding water, and then performing re-distilling to distill a medical solution; then again adding water and sodium chloride to distill a medical solution; and mixing the medical solutions obtained last 3 times to obtain a finished traditional Chinese medicine composition;
the traditional Chinese medicine composition being used for eye care and health protection and being applicable to symptoms comprising:
dry eyes, astringent eyes, itchy eyes, eyestrain, foreign body sensation, pterygium, blurred vision, photophobia, epiphora induced by wind, macular degeneration, prevention of myopia, conjunctival concretion, and cataracts.

2. The traditional Chinese medicine composition for tonifying qi and nourishing blood according to claim 1, comprising, by mass, the following components: 12 parts of saffron, 250 parts of Chinese angelica, 200 parts of ginseng, 300 parts of pipewort, and 250 parts of butterflybush flowers.

3. The traditional Chinese medicine composition for tonifying qi and nourishing blood according to claim 1, comprising, by mass, the following components: 8 parts of saffron, 300 parts of Chinese angelica, 250 parts of ginseng, 200 parts of pipewort, and 300 parts of butterflybush flowers.

4. The traditional Chinese medicine composition for tonifying qi and nourishing blood according to claim 1, comprising, by mass, the following components: 10 parts of saffron, 200 parts of Chinese angelica, 300 parts of ginseng, 250 parts of pipewort, and 200 parts of butterflybush flowers.
